(19) United States
(12) Patent Application Publication
(10) Pub. No.: US 2025/0114140 A1
(43) Pub. Date: Apr. 10, 2025

(54) DISTAL JACKET HOLES FOR WIRE THREADING AND ELECTRODE WELD ALIGNMENT

(71) Applicant: Medtronic Ireland Manufacturing Unlimited Company, Dublin 2 (IE)

(72) Inventors: Enrique Estrada, Santa Rosa, CA (US); Paulina Nguyen, Santa Rosa, CA (US); Kelsey Sandquist, Cotati, CA (US); Cian Michael Ryan, Santa Rosa, CA (US); Sina Som, Santa Rosa, CA (US); Kevin Mauch, Windsor, CA (US); William Chang, Santa Rosa, CA (US)

(21) Appl. No.: 18/687,213

(22) PCT Filed: Sep. 16, 2022
(86) PCT No.: PCT/EP2022/075778

§ 371 Date: Feb. 27, 2024

Related U.S. Application Data
(60) Provisional application No. 63/250,810, filed on Sep. 30, 2021.

(51) Int. Cl. A61B 18/14; A61B 18/00

(52) U.S. Cl. CPC A61B 18/1492; A61B 2018/00077; A61B 2018/00178; A61B 2018/00184; A61B 2018/1475

(57) ABSTRACT

In some examples, a neuromodulation catheter includes a neuromodulation element convertible between a low-profile delivery state and a radially expanded deployed state, the neuromodulation element including: an elongated structure configured to have a substantially linear shape defining a longitudinal axis when the neuromodulation element is in the low-profile delivery state, and further configured to have a coiled shape defining a coiled outer surface when the neuromodulation element is in the radially expanded deployed state; one or more electrodes spaced longitudinally apart along the longitudinal axis of the elongated structure; and one or more wires coupled to respective electrodes of one or more electrodes at respective coupling points, wherein the coupling points are arranged such that, when the neuromodulation element is in the deployed state, the coupling points are oriented along the coiled outer surface of the elongated structure.

### TECHNICAL FIELD

The present technology is related to catheters, and in particular, to neuromodulation catheters including neuromodulation elements configured to deliver energy to nerves at or near a treatment location within a body lumen.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. Excessive activation of the renal SNS, in particular, has been identified experimentally and in humans as a likely contributor to the complex pathophysiologies of hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function are considerably stimulated in disease states characterized by heightened sympathetic tone. For example, reduced renal blood flow and glomerular filtration rate as a result of renal sympathetic efferent stimulation is likely a cornerstone of the loss of renal function in cardio-renal syndrome (i.e., renal dysfunction as a progressive complication of chronic heart failure). Pharmacologic strategies to thwart the consequences of renal sympathetic stimulation include centrally-acting sympatholytic drugs, beta blockers (e.g., to reduce renin release), angiotensin-converting enzyme inhibitors and receptor blockers (e.g., to block the action of angiotensin II and aldosterone activation consequent to renin release), and diuretics (e.g., to counter renal sympathetic mediated sodium and water retention). These pharmacologic strategies, however, have significant limitations including limited efficacy, compliance issues, side effects, and others.

### SUMMARY

The present technology is directed to devices, systems, and methods for neuromodulation, such as renal neuromodulation using radiofrequency (RF) energy. A catheter (e.g., an RF ablation catheter) may be configured to deliver RF energy circumferentially around a lumen (e.g., a renal main artery, accessory renal artery, or branch vessel) in which the catheter is positioned. The catheter may include at least a proximal portion and a distal portion. The distal portion may include one or more electrodes. The distal portion of the catheter may further include one or more wires or wire pairs (e.g., one wire or wire pair for each electrode) running distally through the catheter, each wire or wire pair extending through a corresponding opening or slot. Via the slot, each wire or wire pair electrically couples to a respective electrode at a respective coupling point in order to deliver energy, and in some cases, to form a thermocouple for conducting temperature measurements. The distal portion of the catheter may be configured to transform between a substantially straight delivery configuration and a spiral or coiled deployed configuration.

In examples described herein, the relative positions and orientations of the electrode(s), the coupling point(s), and/or the slot(s) may be selected such that, in the deployed configuration of the catheter, the coupling point(s) are oriented along an outer circumferential surface defined by the coil shape. In this way, the coupling points may be more-precisely positioned against a vessel wall of the patient, enabling more-accurate energy delivery and/or temperature measurements in order to better-inform energy generation during the neuromodulation procedure, thereby improving a likelihood of success of the denervation therapy.

In some examples, a catheter includes a neuromodulation element convertible between a low-profile delivery state and a radially expanded deployed state, the neuromodulation element comprising: an elongated structure configured to have a substantially linear shape defining a longitudinal axis when the neuromodulation element is in the low-profile delivery state, and further configured to have a coiled shape defining a coiled outer surface when the neuromodulation element is in the radially expanded deployed state; one or more electrodes spaced longitudinally apart along the longitudinal axis of the elongated structure; and one or more wires, each wire electrically coupled to a corresponding electrode of the one or more electrodes at a respective coupling point of one or more coupling points, wherein the coupling points are arranged such that, when the neuromodulation element is in the deployed state, the one or more coupling points are oriented along the coiled outer surface of the coiled shape of the elongated structure.

In some examples, a method of forming a neuromodulation element includes: forming a tubular elongated structure, an outer surface of the elongated structure defining one or more reduced-diameter segments spaced apart along a longitudinal axis of the elongated structure; forming a slot of one or more slots within each of the one or more reduced-diameter segments of the elongated structure, the one or more slots positioned such that, when the elongated structure transitions from a generally linear delivery state to a coiled deployed state defining a coil shape having a coiled outer surface, the one or more slots are positioned along the coiled outer surface of the coil shape; extending each of one or more wires or wire pairs through a respective slot of the one or more slots; positioning each of one or more electrodes in a respective reduced-diameter segment of the one or more reduced diameter segments; and electrically coupling each of the one or more wires or wire pairs to a respective one of the one or more electrodes.

This disclosure also describes examples of methods of using the aspiration systems and devices. The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Reference is made to the attached drawings, wherein elements having the same reference numeral designations represent similar elements throughout.

FIG. 1 is a partially schematic perspective view illustrating a therapeutic system configured in accordance with an example of the present technology. The system is shown in FIG. 1 including a neuromodulation catheter having a shaft.

FIG. 2 is an exploded profile view of the catheter shown in FIG. 1.

FIG. 3 is an enlarged exploded profile view of portions of the catheter shown in FIG. 1 taken at respective locations designated in FIG. 2.

FIG. 4 is a perspective view of a distal jacket of a neuromodulation element of a neuromodulation catheter configured in accordance with an example of the present technology. The distal jacket is shown in FIG. 4 including reduced-diameter segments.

FIG. 5 is a profile view of the distal jacket shown in FIG. 4 and band electrodes respectively seated in the reduced-diameter segments.

FIG. 6 is a profile view of the distal jacket shown in FIG. 4.

FIG. 7 is an enlarged profile view of a portion of a distal jacket of the shaft shown in FIG. 1 taken at a location designated in FIG. 6.

FIG. 8 is a cross-sectional profile view of the distal jacket shown in FIG. 5 taken along a line 14-14 designated in FIG. 6.

FIGS. 9-11 are enlarged cross-sectional profile views of a portion of the distal jacket shown in FIG. 4 at a location designated in FIG. 8. The portion of the distal jacket shown in FIGS. 9-11 includes one of the reduced-diameter segments shown in FIG. 4. In FIG. 9, the portion of the distal jacket is shown without a band electrode. In FIG. 10, the portion of the distal jacket is shown resiliently deformed inwardly as a band electrode is moved toward the reduced-diameter segment. In FIG. 9, the portion of the distal jacket is shown with the band electrode seated in the reduced-diameter segment.

FIG. 12 illustrates an example neuromodulation element of a catheter in a linear, delivery configuration.

FIG. 13 illustrates an example of the neuromodulation element of FIG. 12 in a coiled, deployed configuration.

FIG. 14 is a flow chart illustrating a method for making a neuromodulation element in accordance with an example of the present technology.

### DETAILED DESCRIPTION

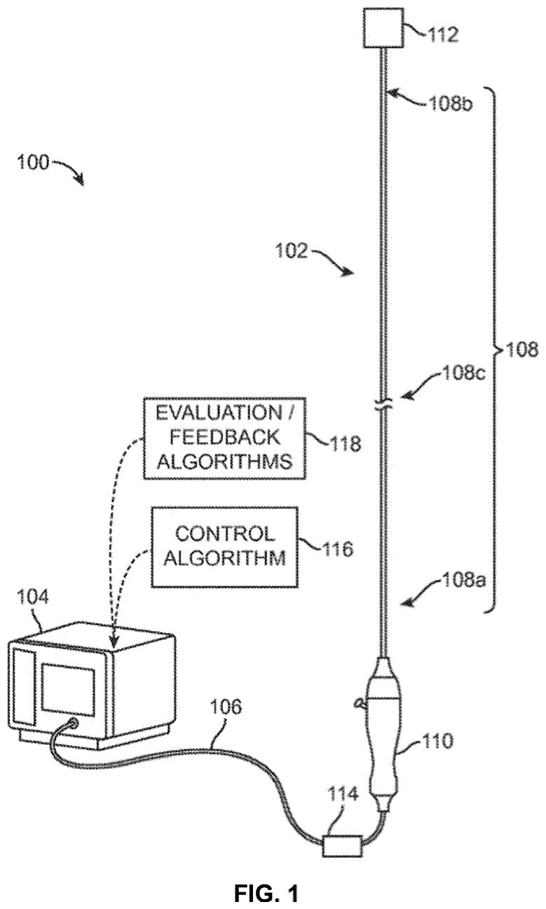

Specific details of systems, devices, and methods in accordance with several examples of the present technology are disclosed herein with reference to FIGS. 1-14. Although the systems, devices, and methods may be disclosed herein primarily or entirely with respect to intravascular renal neuromodulation, other applications in addition to those disclosed herein are within the scope of the present technology. For example, systems, devices, and methods in accordance with at least some examples of the present technology may be useful for neuromodulation within a body lumen other than a vessel, for extravascular neuromodulation, for non-renal neuromodulation, and/or for use in therapies other than neuromodulation. Furthermore, it should be understood, in general, that other systems, devices, and methods in addition to those disclosed herein are within the scope of the present technology. For example, systems, devices, and methods in accordance with examples of the present technology can have different and/or additional configurations, components, and procedures than those disclosed herein. Moreover, a person of ordinary skill in the art will understand that systems, devices, and methods in accordance with examples of the present technology can be without one or more of the configurations, components, and/or procedures disclosed herein without deviating from the present technology.

The present technology is directed to devices, systems, and methods for neuromodulation, such as renal neuromodulation, using radiofrequency (RF) energy.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction toward the clinician or clinician's control device.

Renal neuromodulation, such as renal denervation, may be used to modulate activity of one or more renal nerves and may be used to affect activity of the sympathetic nervous system (SNS). In renal neuromodulation, one or more therapeutic elements may be introduced near renal nerves located between an aorta and a kidney of a patient. In some examples, the one or more therapeutic elements may be carried by or attached to a catheter, and the catheter may be introduced intravascularly, e.g., into a renal artery via a brachial artery, femoral artery, or radial artery approach. In other examples, the one or more therapeutic elements may be introduced extravascularly, e.g., using a laparoscopic technique.

Renal neuromodulation can be accomplished using one or more of a variety of treatment modalities, including electrical stimulation, radio frequency (RF) energy, microwave energy, ultrasound energy, a chemical agent, or the like. In some examples, an RF ablation system includes an RF generator configured to generate RF energy and deliver RF energy to tissue via one or more electrodes carried by a catheter and positioned within a lumen of a body of a patient. For example, the lumen may be a vessel, such as a vein or artery. In some examples, the lumen may be a renal artery, such as a main renal artery, an accessory renal artery, a branch vessel, or the like. The RF energy may heat tissue to which the RF energy is directed (which tissue includes one or more renal nerves) and modulate the activity of the one or more renal nerves.

The RF ablation system may be configured to deliver RF energy via either a monopolar or bipolar arrangement. In a monopolar arrangement, a return or reference electrode may be paced on a patient's skin, and one or more of the electrodes carried by the catheter may be driven to act as active electrodes, either individually, simultaneously, or sequentially. In a bipolar arrangement, the active and return electrodes may both be carried by or attached to the catheter and introduced within the body of the patient. In some examples, a catheter includes one or more electrodes, and the RF generator and electrical connections between the RF generator and the electrode(s) can be configured for monopolar RF energy delivery, bipolar RF energy delivery, or can be controllable between monopolar RF energy delivery and bipolar RF energy delivery.

In many patients, renal nerves generally follow the renal artery and branch vessels from near the aorta to a kidney. The renal nerves may be present in a wall of the renal artery and/or branch vessels and/or in tissue surrounding the renal artery and/or branch vessels. Because renal nerves may be around the renal artery and/or branch vessels and may include multiple nerves and/or nerve branches, it may be desirable to deliver RF energy circumferentially around the renal artery and/or branch vessels to affect as many renal nerves as possible.

In accordance with examples of the current disclosure, a catheter (e.g., an RF ablation catheter) is configured to deliver RF energy circumferentially around a lumen (e.g., a renal main artery, accessory renal artery, or branch vessel) in which the catheter is positioned. The catheter includes at least a proximal portion and a distal portion. The distal portion may include one or more electrodes (e.g., one electrode, two electrodes, three electrodes, four electrodes, or the like) and may be configured to transform between a substantially straight delivery configuration and a spiral or helical deployed configuration. The catheter may further include one or more wires or wire pairs extending from a proximal end (or from near the proximal end) of the catheter to the electrode(s) at the distal portion of the catheter, each wire (or wire pair) being electrically coupled (e.g., welded or otherwise affixed) to a corresponding electrode to delivery energy, and in some examples, to form a thermocouple for conducting temperature measurements. The distal portion of the catheter may also define one or more openings or slots through which the wire(s) extend in order to contact and electrically couple to the electrode(s). The distal portion of the catheter may be configured to transform between a substantially straight delivery configuration and a spiral or coiled deployed configuration.

In examples described herein, the relative positions and orientations of the electrode(s), the coupling point(s), and/or the slot(s) may be selected such that, in the deployed configuration of the catheter, the coupling points are oriented along an outer circumferential surface defined by the coil shape formed by the catheter. In this way, the coupling points may be more-precisely positioned against a vessel wall of the patient, enabling more-accurate temperature measurements in order to better inform energy generation during the neuromodulation procedure, thereby improving a likelihood of success of the denervation therapy.

FIG. 1 is a partially schematic perspective view illustrating a therapeutic system 100 configured in accordance with some examples of the present disclosure. Therapeutic system 100 includes a neuromodulation catheter 102, an RF generator 104, and a cable 106 extending between catheter 102 and RF generator 104. Neuromodulation catheter 102 includes an elongate shaft (also referred to as an elongate body) 108 having a proximal portion 108a, a distal portion 108b, and an optional intermediate portion 108c between proximal portion 108a and distal portion 108b. Neuromodulation catheter 102 may further include a handle 110 operably connected to shaft 108 via proximal portion 108a and a neuromodulation element 112 (shown schematically in FIG. 1 that is part of or attached to distal portion 108b. Shaft 108 is configured to locate the neuromodulation element 112 at a treatment location within or otherwise proximate to a body lumen (e.g., a blood vessel, a duct, an airway, or another naturally occurring lumen within the human body). In some examples, shaft 108 is configured to locate neuromodulation element 112 at an intraluminal (e.g., intravascular) location. Neuromodulation element 112 may be configured to provide or support a neuromodulation treatment at the treatment location. Shaft 108 and neuromodulation element 112 may measure 2, 3, 4, 5, 6, or 7 French or other suitable sizes.

Intraluminal delivery of neuromodulation catheter 102 may include percutaneously inserting a guidewire (not shown) into a body lumen of a patient and moving shaft 108 and neuromodulation element 112 along the guidewire until neuromodulation element 112 reaches a suitable treatment location. Alternatively, neuromodulation catheter 102 may be a steerable or non-steerable device configured for use without a guidewire. Additionally, or alternatively, neuromodulation catheter 102 may be configured for use with another type of guide member, such as a guide catheter or a sheath (not shown), alone, or in addition to a guidewire.

RF generator 104 is configured to control, monitor, supply, and/or otherwise support operation of neuromodulation catheter 102. In other examples, neuromodulation catheter 102 may be self-contained or otherwise configured for operation independent of RF generator 104. When present, RF generator 104 is configured to generate a selected form and/or magnitude of RF energy for delivery to tissue at a treatment location via neuromodulation element 112. For example, RF generator 104 can be configured to generate RF energy (e.g., monopolar and/or bipolar RF energy). In other examples, RF generator 104 may be another type of device configured to generate and deliver another suitable type of energy to neuromodulation element 112 for delivery to tissue at a treatment location via electrodes (not shown) of neuromodulation element 112.

Along cable 106 or at another suitable location within therapeutic system 100, therapeutic system 100 may include a control device 114 configured to initiate, terminate, and/or adjust operation of one or more components of neuromodulation catheter 102 directly and/or via RF generator 104. RF generator 104 may be configured to execute an automated control algorithm 116 and/or to receive control instructions from an operator. Similarly, in some implementations, RF generator 104 is configured to provide feedback to an operator before, during, and/or after a treatment procedure via an evaluation/feedback algorithm 118.

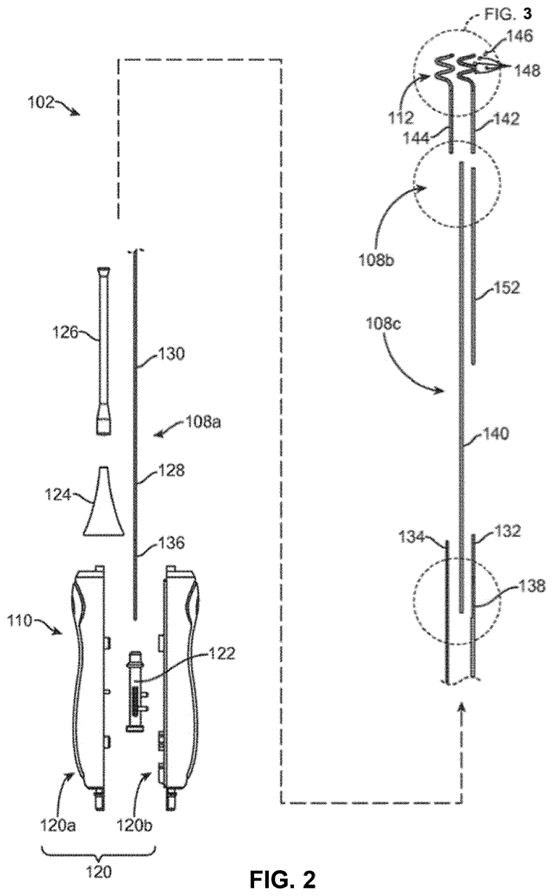

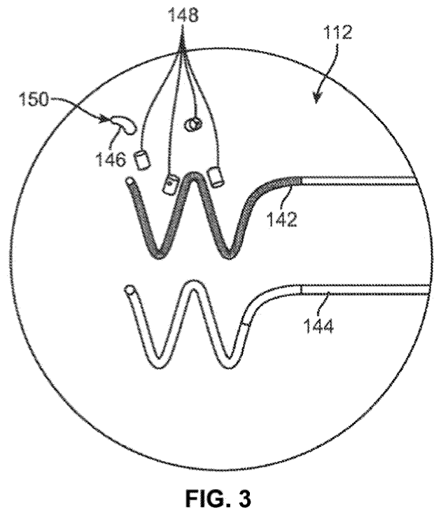

FIG. 2 is an exploded profile view of the catheter 102. FIG. 3 is an enlarged exploded profile view of portions of the catheter 102 taken at the location designated in FIG. 2. With reference to FIG. 3, the handle 110 can include mating shell segments 120 (individually identified as shell segments 120a, 120b) and a connector 122 (e.g., a luer connector) operably positioned between the mating shell segments 120. The handle 110 can further include a distally tapered strain-relief element 124 operably connected to distal ends of the shell segments 120. Slidably positioned over the shaft 108, the catheter 102 can include a loading tool 126 configured to facilitate loading the catheter 102 onto a guidewire (not shown). When assembled, the shaft 108 can extend through coaxial lumens (also not shown) of the strain-relief element 124 and the loading tool 126, respectively, and between the shell segments 120 to the connector 122.

The shaft 108 can include an assembly of parallel tubular segments. At its proximal end portion 108a and extending distally though a majority of its intermediate portion 108c, the shaft 108 can include a proximal hypotube segment 128, a proximal jacket 130, a first electrically insulative tube 132, and a guidewire tube 134. The first electrically insulative tube 132 and the guidewire tube 134 can be disposed side-by-side within the proximal hypotube segment 128. The first electrically insulative tube 132 can be configured to carry electrical leads (not shown) and to electrically insulate the electrical leads from the proximal hypotube segment 128. The guidewire tube 134 can be configured to carry a guidewire (not shown). The proximal jacket 130 can be disposed around at least a portion of an outer surface of the proximal hypotube segment 128. The proximal hypotube segment 128 can include a proximal stem 136 at its proximal end and a distal skive 138 at its distal end.

With reference again to FIGS. 2 and 3, the first electrically insulative tube 132 and the guidewire tube 134 can extend distally beyond the distal skive 138. The shaft 108 can include an intermediate tube 140 beginning proximally at a region of the shaft 108 at which the first electrically insulative tube 132 and the guidewire tube 134 distally emerge from the proximal hypotube segment 128. The intermediate tube 140 can be more flexible than the proximal hypotube segment 128. At the region of the shaft 108 at which the first electrically insulative tube 132 and the guidewire tube 134 distally emerge from the proximal hypotube segment 128, the intermediate tube 140 can be coaxially aligned with the proximal hypotube segment 128 so as to receive the first electrically insulative tube 132 and the guidewire tube 134. From this region, the intermediate tube 140 can extend distally to the distal end portion 108b of the shaft 108. The first electrically insulative tube 132 can distally terminate within the intermediate tube 140. In contrast, the guidewire tube 134 can extend through the entire length of the intermediate tube 140. At a distal end of the intermediate tube 140, the shaft 108 can be operably connected to the neuromodulation element 112.

In FIGS. 2 and 3, the neuromodulation element 112 is shown in a radially expanded deployed state. The neuromodulation element 112 can be movable from a low-profile delivery state to the radially expanded deployed state. When the neuromodulation element 112 is in the radially expanded deployed state, a distal shape-memory structure 142, such as a hypotube (e.g., a cut hypotube), a Nitinol-based structure, or another shape-memory material, can have a shape that is more helical (spiral) than its shape when the neuromodulation element 112 is in the low-profile delivery state. In at least some cases, the distal shape-memory structure 142 has the more helical shape when at rest and is configured to be forced into the less helical shape by an external sheath (not shown). The distal shape-memory structure 142 can be made at least partially of Nitinol, stainless steel, or another suitable material well suited for resiliently moving between the more helical and less helical shapes. In at least some cases, the material of the distal shape-memory structure 142 is electrically conductive. Accordingly, the neuromodulation element 112 can include a second electrically insulative tube 152 disposed around an outer surface of the distal shape-memory structure 142 so as to electrically separate the band electrodes 146 from the distal shape-memory structure 142. In some examples, the first and second electrically insulative tubes 132, 152 are made at least partially (e.g., predominantly or entirely) of polyimide and polyether block amide, respectively. In other examples, the first and second electrically insulative tubes 132, 152 can be made of other suitable materials.

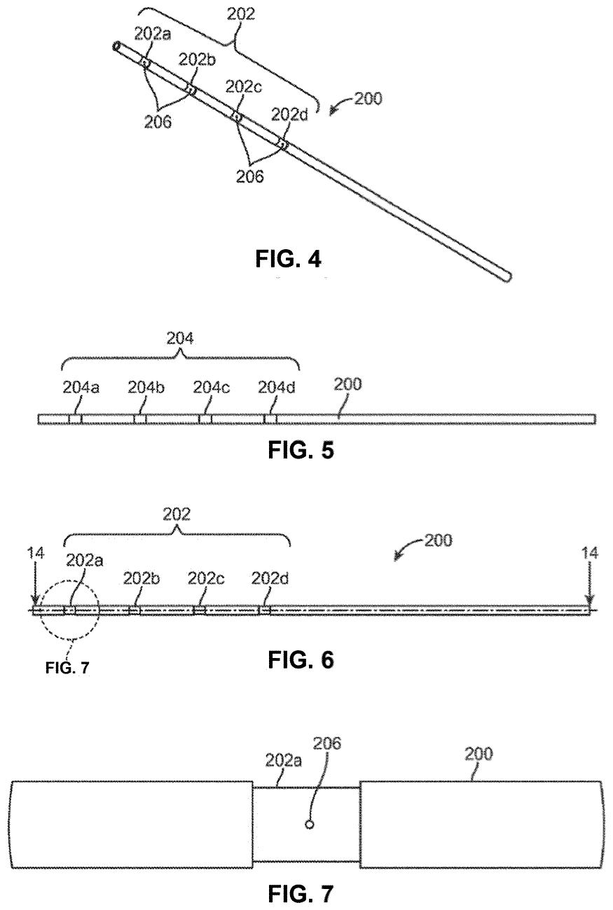

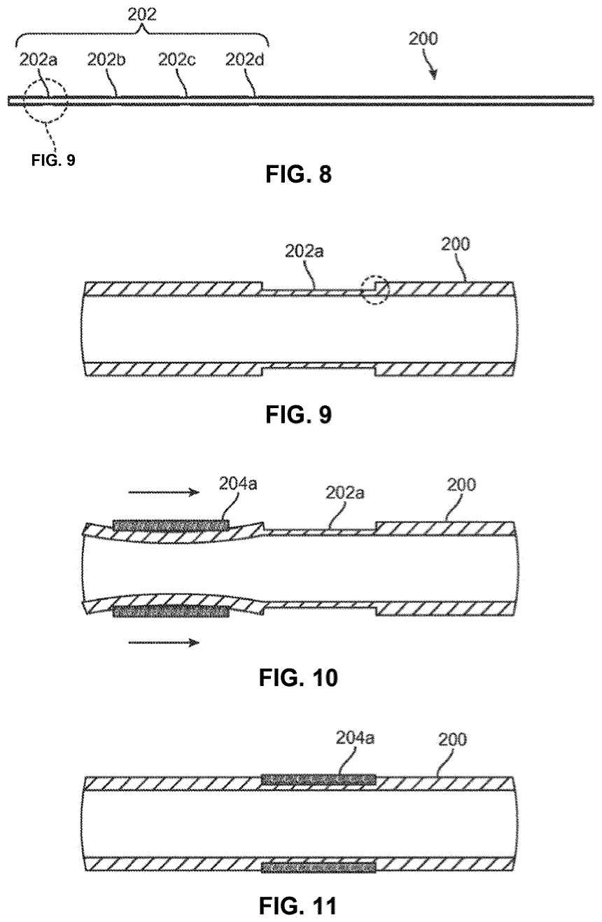

FIG. 4 is a perspective view of a distal jacket 200 of a neuromodulation element of a neuromodulation catheter configured in accordance with an example of the present technology. The distal jacket 200, for example, can be used in the neuromodulation element 112 (FIGS. 1, 2, and 3) in place of the distal jacket 144 (FIGS. 2 and 3). Accordingly, the distal jacket 200 may be described below in conjunction with components of the catheter 102 (FIGS. 1 and 2). The distal jacket 200 can include reduced-diameter segments 202 (individually identified as reduced-diameter segments 202a-202d) extending through its outer surface. FIG. 5 is a profile view of the distal jacket 200 and band electrodes 204 (individually identified as band electrodes 204a-204d) respectively seated in the reduced-diameter segments 202. FIG. 6 is a profile view of the distal jacket 200 without the band electrodes 204. FIG. 7 is an enlarged profile view of a portion of the distal jacket 200 taken at a location designated in FIG. 6. FIG. 8 is a cross-sectional profile view of the distal jacket 200 taken along a line 14-14 designated in FIG. 6.

With reference to FIGS. 4-8 together, the distal jacket 200 can be tubular and configured to be disposed around at least a portion of an outer surface of the distal shape-memory structure 142 (FIGS. 2 and 3). The reduced-diameter segments 202 can be insets, pockets, grooves, or other suitable features configured to respectively seat the band electrodes 204. In the illustrated example, the distal jacket 200 includes exactly four reduced-diameter segments 202 spaced apart along its longitudinal axis. Alternatively, the distal jacket 200 can include exactly one, two, three, five, six or a greater number of reduced-diameter segments 202. The reduced-diameter segments 202 may be spaced apart at equal distances or at different distances. The distal jacket 200 can include one or more openings 206 respectively positioned at the reduced-diameter segments 202. A neuromodulation catheter including the distal jacket 200 can include electrical leads (not shown) extending from respective reduced-diameter segments 202, through respective openings 206, through a lumen of the distal shape-memory structure 142 (FIGS. 2 and 3), through the intermediate tube 140, and through the proximal hypotube segment 128 to the handle 110. In this way, the electrical leads can respectfully connect the band electrodes 204 to proximal components of a neuromodulation catheter including the distal jacket 200.

FIGS. 9-11 are enlarged cross-sectional profile views of a portion of the distal jacket 200 at a location designated in FIG. 8. At this location, the distal jacket 200 can include the reduced-diameter segment 202a. In FIG. 9, the portion of the distal jacket 200 is shown without the band electrode 204a corresponding to the reduced-diameter segment 202a. In FIG. 10, the portion of the distal jacket 200 is shown resiliently deformed inwardly as the band electrode 204a is moved toward the reduced-diameter segment 202a. In FIG. 11, the portion of the distal jacket 200 is shown with the band electrode 204 a seated in the reduced-diameter segment 202a. With reference to FIGS. 9-11 together, the band electrodes 204 can respectively form closed loops extending circumferentially around the distal jacket 200. In at least some cases, a minimum inner diameter of the band electrodes 204 is smaller than a maximum outer diameter of distal jacket 200 between the reduced-diameter segments 202. To facilitate assembly, the distal jacket 200 between the reduced-diameter segments 202 can be resilient in response to peristaltic deflection of a magnitude corresponding to a difference between the maximum outer diameter of the distal jacket 200 between the reduced-diameter segments 202 and the minimum inner diameter of the band electrodes 204. Suitable materials for the distal jacket 200 include polymer blends including polyurethane and polysiloxane, among others.

A maximum outer diameter of the band electrodes 204 and the maximum outer diameter of the distal jacket 200 between the reduced-diameter segments 202 can be at least generally equal (e.g., within 5%, 3%, or 2% of one another). Thus, once the band electrodes 204 are respectively seated in the reduced-diameter segments 202, outer surfaces of the band electrodes 204 and the distal jacket 200 between the reduced-diameter segments 202 can be at least generally flush. This can be useful, for example, to reduce or eliminate potentially problematic ridges (e.g., circumferential steps) at distal and proximal ends of the individual band electrodes 204. This, in turn, can reduce or eliminate the need for fillets (e.g., adhesive fillets, such as glue fillets) at the distal and proximal ends of the individual band electrodes 204. In at least some examples, the distal jacket 200 and the band electrodes 204 can be bonded to one another without any exposed adhesive. For example, an adhesive (not shown) can be disposed between the band electrodes 204 and the distal jacket 200 at the reduced-diameter segments 202.

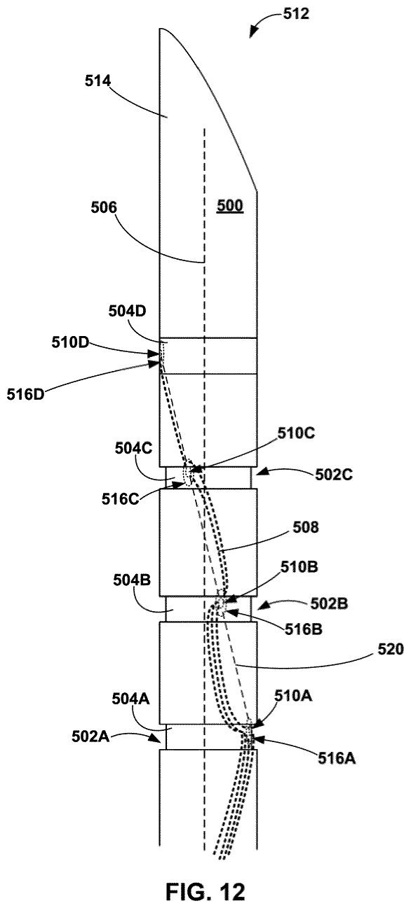

FIG. 12 depicts an example neuromodulation element 512 in a generally linear, “delivery” state configured for introduction into a vasculature of a patient. Neuromodulation element 512 is an example of neuromodulation element 112 of FIGS. 1, 2, and 3, and accordingly, may be described below in conjunction with components of catheter 102 (FIGS. 1 and 2). Neuromodulation element 512 includes an elongated structure 500. As described above, elongated structure 500 can include an outer jacket 514 (e.g., distal jacket 200) and an elongated shape-memory structure (e.g., distal shape-memory structure 142) positioned within an inner lumen of the jacket. In some examples, the elongated shape-memory structure may include a hypotube, such as a cut hypotube, and/or a spiral-shaped Nitinol coil, such as a helical hollow strand (HHS) structure. In some such examples, the HHS structure can include a plurality of wires or filaments braided or otherwise interwoven into a coil shape, and then physically or chemically treated to impart desired shape-memory properties. In some examples, the outer jacket can include a polymer blend including polyurethane, polyether block amide, and/or polysiloxane.

As described above with respect to distal jacket 200, elongated structure 500 defines an inner lumen and one or more reduced-diameter segments 502 (individually identified in FIG. 12 as reduced-diameter segments 502A-502C) spaced longitudinally apart, e.g., in a distal direction (bottom-to-top, from the perspective of FIG. 12) along central longitudinal axis 506 of elongated structure 500.

Neuromodulation element 512A further includes one or more electrodes 504 (individually identified in FIG. 12 as electrodes 504A-504D). Some, but not necessarily all, of electrodes 504 can include ring or band electrodes. In the example of FIG. 12, electrodes 504A-504C are respectively seated in the reduced-diameter segments 502A-502C, whereas distal-most electrode 504D is not seated within a reduced-diameter segment 502. As shown in FIG. 12, elongated structure 500 may include a tapered distal end (e.g., a distal end oriented at an oblique angle relative to central longitudinal axis 506) to facilitate placement of electrodes 504 overtop of elongated structure 500 via the distal end. In some examples, but not all examples, neuromodulation element 512 includes a plurality of glue fillets configured to provide a smoother outer surface for neuromodulation element 512, e.g., adjacent to proximal and distal edges of electrodes 504 to transition between electrodes 504 and outer jacket 514.

As shown in FIG. 12, neuromodulation element 512 includes one or more wires or wire pairs 508 (collectively, “wire(s) 508”), e.g., one wire or wire pair 508 for each electrode 504. For instance, neuromodulation element 512 may include one or more individual wires, formed from copper, gold, silver, platinum, or the like, for delivering energy to respective electrode(s) 504. In other examples, neuromodulation element 512 may include one or more pairs of wires 508, e.g., each wire pair including one copper wire and one constantan wire, for both delivering energy via respective electrode(s) 504 and also for sensing a temperature at respective electrode(s) 504.

Wire(s) 508 extend generally distally (e.g., in a bottom-to-top direction, from the perspective of FIG. 12) along the length of the neuromodulation element 512, e.g., through the inner lumen of elongated structure 500. For instance, in examples in which elongated structure 500 includes both an outer jacket and an inner shape-memory structure, wire(s) 508 may be positioned radially outward from the shape-memory structure and radially inward from the outer jacket, or in other words, within an annular-shaped lumen defined by or within elongated structure 500. While the neuromodulation element is in the generally linear (e.g., low-profile) “delivery” configuration depicted in FIG. 12, wire(s) 508 extend generally distally along longitudinal axis 506.

In some examples, wire(s) 508 run in parallel, and each wire or wire pair 508 is electrically coupled to one electrode 504. For instance, as shown in FIG. 12, four pairs of wire(s) 508 run in parallel along a segment proximal to electrode 504A. One of those four wire pairs 508 couples to electrode 504A and terminates at electrode 504A, such that three wire pairs 508 extend distally past electrode 504A toward electrode 504B. One of those three wire pairs 508 couples to, and terminates at, electrode 504B, such that two wire pairs 508 extend distally past electrode 504B toward electrode 504C. One of those two wire pairs 508 couples to, and terminates at, electrode 504C, such that only one wire pair 508 extends distally past electrode 504C to couple to, and terminate at, electrode 504D.

Elongated structure 500 (e.g., an outer jacket of elongated structure 500) further defines one or more openings or “slots” 516A-516D extending radially through the material of elongated structure 500. In some examples, slots 516 are positioned at least in respective reduced-diameter segments 502A-502C, and in the example shown in FIG. 12, at a position distal to reduced-diameter segment 502C. Each wire or wire pair 508 extends through one of slots 516 and is electrically coupled (e.g., welded, soldered, etc.) to a respective electrode 504A-504D, thereby forming a respective coupling point 510A-510D. In examples in which the neuromodulation element 512 includes wire pairs 508 (as opposed to just lone wires), coupling points 510 function as thermocouples for sensing (e.g., measuring) a temperature surrounding the respective electrode 504. These temperature measurements may be used to inform a required amount of power to be generated (e.g., by RF generator 104 of FIG. 1) during a neuromodulation procedure.

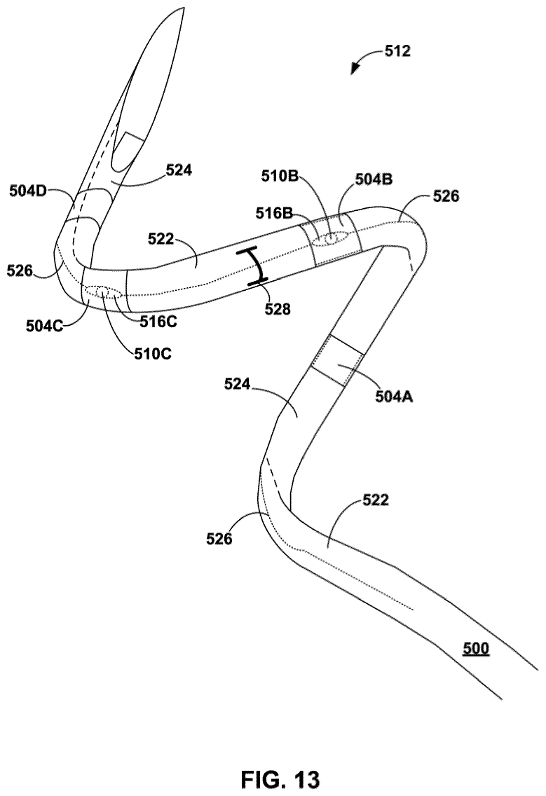

In examples described herein, the relative positions and/or orientations of any or all of electrode(s) 504, coupling point(s) 510 between wire(s) 508 and electrode(s) 504, and slot(s) 516 may be selected such that, in the radially expanded (e.g., helical or coiled) deployed state of neuromodulation element 512 (e.g., as shown in FIG. 13), the coupling point(s) 510 are generally oriented along a coiled exterior surface defined by the coil shape. In this way, the coupling point(s) 510 may be more-precisely positioned against the vessel wall of the patient, enabling more-efficient energy delivery, and in appropriate examples, more-accurate temperature measurements to better-inform energy generation during the neuromodulation procedure, thereby improving a likelihood of success of the denervation therapy and improving patient outcomes.

In the example configuration depicted in FIG. 12, coupling points 510 and jacket slots 516 are mutually oriented along a line 520 that is not parallel to longitudinal axis 506 of neuromodulation element 512. For instance, orientation line 520 may extend in both a longitudinal (e.g., distal) direction along longitudinal axis 506 of neuromodulation element 512, as well as circumferentially around elongated structure 500, such that coupling points 510 and jacket slots 516 collectively extend helically around elongated structure 500.

Such helical orientations or distributions of coupling points 510 and jacket slots 516 around elongated structure 500 (e.g., while elongated structure 500 is in a generally low-profile, linear state) could result in the coupling points 510 (e.g., thermocouples) being more-accurately oriented around an outer or exterior coiled surface of a coiled shape defined by elongated structure 500 when elongated structure 500 transitions to the radially expanded, deployed configuration. For instance, depending on a plurality of size and shape parameters of neuromodulation element 512, including, as non-limiting examples, the dimensions (e.g., radius, axial length) of the expanded coil shape, the axial spacing of electrodes 504, the number of “turns” of the expanded coil shape, or the like, a helical-type distribution of jacket slots 516 and coupling points 510 can help ensure that the coupling points 510 are positioned correctly in the subsequent coiled configuration of neuromodulation element 512. As one non-limiting example of these parameters and dimensions, adjacent jacket slots 516 may be spaced longitudinally apart by about 5 mm along longitudinal axis 506, and spaced circumferentially apart by about 60 degrees around longitudinal axis 506.

The example orientations of coupling points 510 and jacket slots 516 depicted in FIG. 12 are not intended to be limiting. As described above, the relative positions and orientations of electrodes 504, coupling points 510, and jacket slots 516 may be selected in accordance with the techniques of this disclosure such that, when neuromodulation element 512 transitions from a delivery configuration (as depicted in FIG. 12) to a radially expanded deployed configuration, thermocouple weld points 510 are oriented along an external surface of the shape formed by neuromodulation element 512. In examples in which neuromodulation element 512 includes only a single electrode 504, a single coupling point 510, and a single jacket slot 516, the relative positions of these three elements can nevertheless be selected such that the single coupling point 510 is positioned on an outer circumferential surface of neuromodulation 512 while in the “deployed” configuration.

For instance, as shown in FIG. 13, while in a “deployed” configuration, the shape-memory structure of elongated structure 500 may cause neuromodulation element 512 to assume the form of a radially expanded coil, defining a coiled “outer” surface 522 and a coiled “inner” surface 524. While deployed within a vessel of a patient, coiled outer surface 522 is configured to contact the patient's vessel wall. When the coupling point(s) 510 between the wire(s) 508 and electrode(s) 504 are oriented along outer surface 522 of the coil, the coupling points may be used to deliver energy directly onto the blood vessel wall of the patient. In some such examples in which the wire(s) 508 include wire pair(s), the thermocouples defined by coupling points 510 may be used to sense a temperature of the blood vessel wall of the patient, which in turn may be used by RF generator 104 (FIG. 1) to inform an amount of power to generate.

In particular, locations along the exact center of coiled outer surface 522 collectively define an “origin” line 526 that extends helically around neuromodulation element 512. Any such location along origin line 526 may be referred to as a “zero degree” position. In accordance with some example techniques of this disclosure, the coupling point(s) 510 (as well as corresponding slot(s) 516) are aligned and oriented with respect to one another such that the coupling points 510 and jacket slots 516 are positioned within a threshold arc length 528 from origin line 526 while neuromodulation element 512 is in the deployed state. In some cases, the threshold arc length 528 may extend a maximum of about 90 degrees (e.g., around a circumference of elongated structure 500) on either circumferential side of origin line 528. In some such examples, the threshold arc length 528 may extend about 45 degrees on either circumferential side of origin line 528. In some such examples, the threshold arc length 528 may extend a maximum of about 22.5 degrees on either circumferential side of origin line 528. While the exact orientation of coupling points 510 and slots 516 may be variably selected based on, for example, the number and longitudinal spacing of electrodes 504, the radius of the coil shape formed by neuromodulation element 512 in the deployed state, in some examples, a generally helical orientation of coupling points 510 (e.g., spiraling distally along elongated structure 500), such as that depicted in FIG. 12, may be selected to conform to the threshold arc length 528.

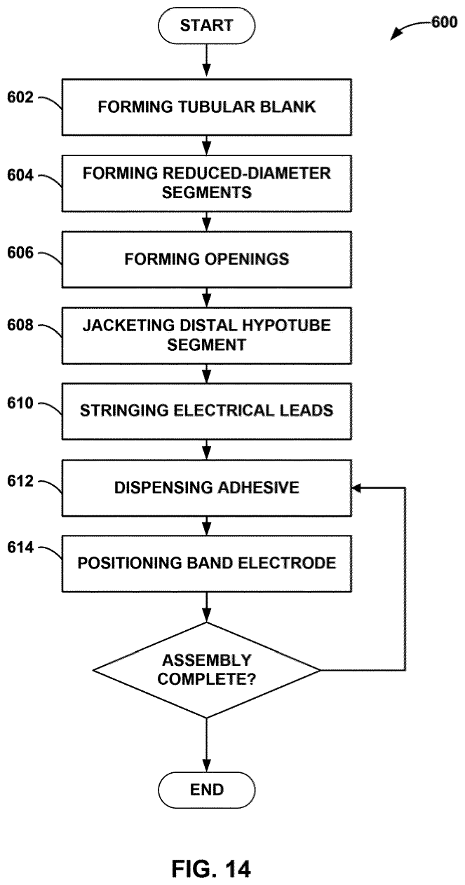

FIG. 14 is a flow chart illustrating a method 600 for making a neuromodulation element including, the distal jacket 200 and the band electrodes 204 in accordance with an example of the present technology. With reference to FIGS. 4-13 together, the method 600 can begin with forming the distal jacket 200 (e.g., elongated structure 500 of FIGS. 12 and 13). This can include forming a tubular blank (block 602) (e.g., by extrusion).

The method 600 then includes using a subtractive process (e.g., by laser ablation) to remove portions of the blank and thereby form one or more reduced-diameter segments 202 (e.g., segment(s) 502 of FIGS. 12 and 13) (block 604), and using the same or a different subtractive process to form one or more openings 206 (e.g., jacket slot(s) 516 of FIGS. 12 and 13) (block 606). Block 606 of the method 600 may be performed either before or after block 604. As described above, the subtractive process used to form openings 206 (slot(s) 516) can be precisely controlled such that openings 206 are formed at relative positions that face radially outward when distal jacket 200 (e.g., elongated structure 500) transitions from the linear delivery state to the radially expanded state.

Alternatively, the distal jacket 200 can be formed by injection molding or another suitable technique that allows the reduced-diameter segments 202 and/or the openings 206 to be formed without the need for a subtractive process. When a subtractive process is used to form the reduced-diameter segments 202, the subtractive process can be precisely controlled so as to leave an innermost portion of a wall of the distal jacket 200 intact at the reduced-diameter segments 202. Laser ablation is one example of a suitable subtractive process for forming the reduced-diameter segments 202. Laser ablation can include loading the blank onto a mandrel and then rotating the blank and the mandrel relative to an ablative laser (or rotating the ablative laser relative to the black and the mandrel) under computerized control. The mandrel can conductively cool the innermost portion of the wall of the distal jacket 200 so as to prevent this portion of the wall from reaching ablative temperatures at the reduced-diameter segments 202. Furthermore, laser ablation and other subtractive processes can be carefully controlled to avoid forming a notch or other indentation in the distal jacket 200 below the floor 206 at the corner 210. When present, such an indentation may unduly decrease the tensile strength of the distal jacket 200. Other techniques for forming the reduced-diameter segments 202 are also possible.

The method 600 can further include jacketing the distal shape-memory structure 142 (block 608) and stringing electrical leads (e.g., the one or more wires or wire pairs 508 of FIGS. 12 and 13) (block 610) from the reduced-diameter segments 202 through a lumen of the distal shape-memory structure 142. Block 610 of the method 600 may be performed either before or after block 608. For example, jacketing the distal shape-memory structure 142 (608) may be performed by positioning the distal jacket 200 and the distal shape-memory structure 142 relative to one another so that the distal jacket 200 is disposed around at least a portion of an outer surface of the distal shape-memory structure 142. In at least some examples, the form and/or other aspects of the distal jacket 200 may allow the distal jacket 200 to be disposed around at least a portion of the outer surface of the distal shape-memory structure 142 without swaging the distal jacket 200.

In some examples, but not all examples, the method 600 can include dispensing an adhesive (block 612) onto the distal jacket 200 at the reduced-diameter segment 202d and positioning the band electrode 204d (e.g., electrode 504A of FIGS. 12 and 13) (block 614) at the reduced-diameter segment 202d (e.g., segment 502A of FIGS. 12 and 13). Block 614 of the method 600 may be performed either before or after block 612. For instance, in examples in which a chemical adhesive is applied, and/or in which the band electrode 204d is not fully circumferential, the electrode 204d can be positioned either before or after applying the adhesive, e.g., from the “side” of the distal jacket 200 rather than over a distal tip of the distal jacket 200. In other examples, an adhesive material may not be used, and instead, the band electrode 204d may be secured in place at the reduced-diameter segment by other retention means.

As discussed above with reference to FIGS. 9-11, positioning the band electrode 204d can include resiliently deforming the distal jacket 200 inwardly while passing (e.g., advancing or threading) the distal jacket 200 through a channel of the band electrode 204d so as to move the band electrode 204d toward a longitudinal position at which the band electrode 204d is aligned with the reduced-diameter segment 202d. The same process can be used to install the band electrodes 204c, the band electrode 204b, and finally the band electrode 204a.

Catheters configured in accordance with at least some examples of the present technology can be well suited (e.g., with respect to sizing, flexibility, operational characteristics, and/or other attributes) for performing renal neuromodulation in human patients. Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically-induced, thermally-induced, or induced in another suitable manner or combination of manners at one or more suitable treatment locations during a treatment procedure. The treatment location can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a treatment procedure can include modulating nerves in the renal plexus, which lay intimately within or adjacent to the adventitia of the renal artery. Various suitable modifications can be made to the catheters described above to accommodate different treatment modalities. For example, the band electrodes 204 can be replaced with transducers to facilitate transducer-based treatment modalities.

Renal neuromodulation can include an electrode-based treatment modality alone or in combination with another treatment modality. Electrode-based treatment can include delivering electricity and/or another form of energy to tissue at or near a treatment location to stimulate and/or heat the tissue in a manner that modulates neural function. For example, sufficiently stimulating and/or heating at least a portion of a sympathetic renal nerve can slow or potentially block conduction of neural signals to produce a prolonged or permanent reduction in renal sympathetic activity. A variety of suitable types of energy can be used to stimulate and/or heat tissue at or near a treatment location. For example, neuromodulation in accordance with examples of the present technology can include delivering RF energy and/or another suitable type of energy. An electrode used to deliver this energy can be used alone or with other electrodes in a multi-electrode array.

Heating effects of electrode-based treatment can include ablation and/or non-ablative alteration or damage (e.g., via sustained heating and/or resistive heating). For example, a treatment procedure can include raising the temperature of target neural fibers to a target temperature above a first threshold to achieve non-ablative alteration, or above a second, higher threshold to achieve ablation. The target temperature can be higher than about body temperature (e.g., about 37° C.) but less than about 45° C. for non-ablative alteration, and the target temperature can be higher than about 45° C. for ablation. Heating tissue to a temperature between about body temperature and about 45° C. can induce non-ablative alteration, for example, via moderate heating of target neural fibers or of luminal structures that perfuse the target neural fibers. In cases where luminal structures are affected, the target neural fibers can be denied perfusion resulting in necrosis of the neural tissue. Heating tissue to a target temperature higher than about 45° C. (e.g., higher than about 60° C.) can induce ablation, for example, via substantial heating of target neural fibers or of luminal structures that perfuse the target fibers. In some patients, it can be desirable to heat tissue to temperatures that are sufficient to ablate the target neural fibers or the luminal structures, but that are less than about 90° C. (e.g., less than about 85° C., less than about 80° C., or less than about 75° C.).

This disclosure is not intended to be exhaustive or to limit the present technology to the precise forms disclosed herein. Although specific examples are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the examples of the present technology. Although steps of methods may be presented herein in a particular order, in alternative examples the steps may have another suitable order. Similarly, certain aspects of the present technology disclosed in the context of particular examples can be combined or eliminated in other examples. Furthermore, while advantages associated with certain examples may have been disclosed in the context of those examples, other examples may also exhibit such advantages, and not all examples need necessarily exhibit such advantages or other advantages disclosed herein to fall within the scope of the present technology. Accordingly, this disclosure and associated technology can encompass other examples not expressly shown and/or described herein.

The methods disclosed herein include and encompass, in addition to methods of practicing the present technology (e.g., methods of making and using the disclosed devices and systems), methods of instructing others to practice the present technology. For example, a method in accordance with a particular example includes forming a tubular jacket, resiliently deforming the jacket inwardly while passing the jacket through a channel of a band electrode, and positioning the jacket and a hypotube segment relative to one another so that the jacket is disposed around at least a portion of an outer surface of the hypotube segment. A method in accordance with another example includes instructing such a method.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one example,” “an example,” or similar formulations means that a particular feature, structure, operation, or characteristic described in connection with the example can be included in at least one example of the present technology. Thus, the appearances of such phrases or formulations herein are not necessarily all referring to the same example. Furthermore, various particular features, structures, operations, or characteristics may be combined in any suitable manner in one or more examples of the present technology.

The techniques described in this disclosure, including those attributed to control circuitry, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example. In addition, analog circuits, components and circuit elements may be employed to construct one, some or all of the control circuitry, instead of or in addition to the partially or wholly digital hardware and/or software described herein. Accordingly, analog or digital hardware may be employed, or a combination of the two. Whether implemented in digital or analog form, or in a combination of the two, control circuitry can comprise a timing circuit.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

The following clauses provide some examples of the disclosure. The examples described herein may be combined in any permutation or combination.

Various aspects of the disclosure have been described. These and other aspects are within the scope of the following claims.

Aspects and embodiments of the invention may be defined by the following clauses.
Clause 1. A catheter comprising a neuromodulation element convertible between a low-profile delivery state and a radially expanded deployed state, the neuromodulation element comprising:
an elongated structure configured to have a substantially linear shape defining a longitudinal axis when the neuromodulation element is in the low-profile delivery state, and further configured to have a coiled shape defining a coiled outer surface when the neuromodulation element is in the radially expanded deployed state;
one or more electrodes spaced longitudinally apart along the longitudinal axis of the elongated structure; and
one or more wires, each wire electrically coupled to a corresponding electrode of the one or more electrodes at a respective coupling point of one or more coupling points, wherein the coupling points are arranged such that, when the neuromodulation element is in the deployed state, the one or more coupling points are oriented along the coiled outer surface of the coiled shape of the elongated structure.
Clause 2. The catheter of clause 1, wherein the one or more coupling points are positioned within a threshold arc length from an origin line defined by a center of the coiled outer surface of the coiled shape of the elongated structure.
Clause 3. The catheter of clause 2, wherein the threshold arc length circumferentially extends about 90 degrees on either side of the origin line.
Clause 4. The catheter of clause 3, wherein the threshold arc length circumferentially extends about 45 degrees on either side of the origin line.
Clause 5. The catheter of clause 3 or clause 4, wherein the threshold arc length circumferentially extends about 22.5 degrees on either side of the origin line.
Clause 6. The catheter of any of clauses 1-5,
wherein the elongated structure comprises a tubular structure defining an inner lumen and one or more slots;
wherein the inner lumen is configured to receive the one or more wires; and
wherein each wire of the one or more wires is configured to extend through a respective slot of the one or more slots and electrically couple to a respective electrode of the one or more electrodes.
Clause 7. The catheter of clause 6, wherein the one or more slots define relative positions of the one or more coupling points, and wherein the relative positions of the one or more coupling points are oriented along the coiled outer surface of the coiled shape of the elongated structure.
Clause 8. The catheter of any of clauses 1-7, wherein the elongated structure comprises a shape memory structure and an outer jacket, and wherein the shape memory structure is pre-formed to urge the neuromodulation element toward the radially expanded deployed state.
Clause 9. The catheter of clause 8, wherein the elongated structure comprises a Nitinol coil.
Clause 10. The catheter of any of clauses 1-9, wherein one or more coupling points comprise at least two coupling points, and wherein the at least two coupling points are arranged such that, when the neuromodulation element is in the low-profile delivery state, the at least two coupling points extend helically around the longitudinal axis of the elongated structure.
Clause 11. The catheter of any of clauses 1-10, wherein the one or more electrodes comprise one or more band electrodes, wherein each band electrode of the one or more band electrodes extends circumferentially around the longitudinal axis of the elongated structure.
Clause 12. The catheter of clause 11,
wherein the elongated structure defines one or more reduced-diameter segments spaced longitudinally apart along the longitudinal axis of the elongated structure; and
wherein each of the one or more band electrodes is seated in a respective reduced-diameter segment of the one or more reduced-diameter segments of the elongated structure.
Clause 13. The catheter of clause 12, wherein the one or more reduced-diameter segments are fully circumferential.
Clause 14. The catheter of any of clauses 1-13, wherein a distal end of the elongated structure is oriented at an oblique angle to the longitudinal axis of the elongated structure.
Clause 15. The catheter of any one of clauses 1 to 14, wherein the one or more electrodes comprise at least three electrodes.
Clause 16. The catheter of clause 15, wherein the catheter includes exactly three electrodes.
Clause 17. The catheter of clause 15, wherein the catheter includes exactly four electrodes.
Clause 18. The catheter of any of clauses 1-17, wherein each wire of the one or more wires comprises a wire pair, and wherein each coupling point of the one or more coupling point comprises a thermocouple point.
Clause 19. The catheter of clause 18, wherein the wire pair comprises a copper wire and a constantan wire.
Clause 20. A method comprising forming a neuromodulation element, wherein forming the neuromodulation element comprises:
forming a tubular elongated structure, an outer surface of the elongated structure defining one or more reduced-diameter segments spaced apart along a longitudinal axis of the elongated structure;
forming a slot of one or more slots within each of the one or more reduced-diameter segments of the elongated structure, the one or more slots positioned such that, when the elongated structure transitions from a generally linear delivery state to a coiled deployed state defining a coil shape having a coiled outer surface, the one or more slots are positioned along the coiled outer surface of the coil shape;
extending each of one or more wires through a respective slot of the one or more slots;
positioning each of one or more electrodes in a respective reduced-diameter segment of the one or more reduced diameter segments; and
electrically coupling each of the one or more wires to a respective one of the one or more electrodes.
Clause 21. The method of clause 20, wherein forming the one or more slots comprises forming the one or more slots at respective one or more positions that collectively extend helically around the longitudinal axis of the elongated structure when the elongated structure is in the linear delivery state.
Clause 22. The method of clause 20 or clause 21, wherein forming the elongated structure includes forming the elongated structure by injection molding.
Clause 23 The method of clause 20 or clause 21, wherein forming the elongated structure includes:
forming a tubular blank by extrusion; and
removing a portion of the blank to form the reduced-diameter segments.
Clause 24. The method of any of clauses 20-23, wherein removing the portion of the blank includes removing the portion of the blank by laser ablation.

1. A catheter comprising a neuromodulation element convertible between a low-profile delivery state and a radially expanded deployed state, the neuromodulation element comprising:
an elongated structure configured to have a substantially linear shape defining a longitudinal axis when the neuromodulation element is in the low-profile delivery state, and further configured to have a coiled shape defining a coiled outer surface when the neuromodulation element is in the radially expanded deployed state;
one or more electrodes spaced longitudinally apart along the longitudinal axis of the elongated structure; and
one or more wires, each wire electrically coupled to a corresponding electrode of the one or more electrodes at a respective coupling point of one or more coupling points, wherein the coupling points are arranged such that, when the neuromodulation element is in the deployed state, the one or more coupling points are oriented along the coiled outer surface of the coiled shape of the elongated structure.

2. The catheter of claim 1, wherein the one or more coupling points are positioned within a threshold arc length from an origin line defined by a center of the coiled outer surface of the coiled shape of the elongated structure.

3. The catheter of claim 2, wherein the threshold arc length circumferentially extends about 90 degrees on either side of the origin line.

4. The catheter of claim 1,
wherein the elongated structure comprises a tubular structure defining an inner lumen and one or more slots;
wherein the inner lumen is configured to receive the one or more wires; and
wherein each wire of the one or more wires is configured to extend through a respective slot of the one or more slots and electrically couple to a respective electrode of the one or more electrodes.

5. The catheter of claim 4, wherein the one or more slots define relative positions of the one or more coupling points, and wherein the relative positions of the one or more coupling points are oriented along the coiled outer surface of the coiled shape of the elongated structure.

6. The catheter of claim 1, wherein the elongated structure comprises a shape memory structure and an outer jacket, and wherein the shape memory structure is pre-formed to urge the neuromodulation element toward the radially expanded deployed state.

7. The catheter of claim 1, wherein one or more coupling points comprise at least two coupling points, and wherein the at least two coupling points are arranged such that, when the neuromodulation element is in the low-profile delivery state, the at least two coupling points extend helically around the longitudinal axis of the elongated structure.

8. The catheter of claim 1, wherein the one or more electrodes comprise one or more band electrodes, wherein each band electrode of the one or more band electrodes extends circumferentially around the longitudinal axis of the elongated structure.

9. The catheter of claim 8,
wherein the elongated structure defines one or more reduced-diameter segments spaced longitudinally apart along the longitudinal axis of the elongated structure; and
wherein each of the one or more band electrodes is seated in a respective reduced-diameter segment of the one or more reduced-diameter segments of the elongated structure.

10. The catheter of claim 1, wherein a distal end of the elongated structure is oriented at an oblique angle to the longitudinal axis of the elongated structure.

11. The catheter of claim 1, wherein the one or more electrodes comprise at least three electrodes.

12. The catheter of claim 11, wherein the catheter includes exactly three electrodes; or wherein the catheter includes exactly four electrodes.

13. The catheter of claim 1, wherein each wire of the one or more wires comprises a wire pair, and wherein each coupling point of the one or more coupling point comprises a thermocouple point.

14. The catheter of claim 13, wherein the wire pair comprises a copper wire and a constantan wire.

15. A method comprising forming a neuromodulation element, wherein forming the neuromodulation element comprises:
forming a tubular elongated structure, an outer surface of the elongated structure defining one or more reduced-diameter segments spaced apart along a longitudinal axis of the elongated structure;
forming a slot of one or more slots within each of the one or more reduced-diameter segments of the elongated structure, the one or more slots positioned such that, when the elongated structure transitions from a generally linear delivery state to a coiled deployed state defining a coil shape having a coiled outer surface, the one or more slots are positioned along the coiled outer surface of the coil shape;
extending each of one or more wires through a respective slot of the one or more slots;
positioning each of one or more electrodes in a respective reduced-diameter segment of the one or more reduced diameter segments; and
electrically coupling each of the one or more wires to a respective one of the one or more electrodes.

16. The method of claim 15, wherein forming the one or more slots comprises forming the one or more slots at respective one or more positions that collectively extend helically around the longitudinal axis of the elongated structure when the elongated structure is in the linear delivery state.

17. The method of claim 15, wherein forming the elongated structure includes forming the elongated structure by injection molding.

18. The method of claim 15, wherein forming the elongated structure includes:
forming a tubular blank by extrusion; and
removing a portion of the blank to form the reduced-diameter segments.

19. The method of claim 18, wherein removing the portion of the blank includes removing the portion of the blank by laser ablation.

20. The catheter of claim 2, wherein the threshold arc length circumferentially extends about 22.5 or about 45 degrees on either side of the origin line.
